(19) United States
(12) Patent Application Publication
(10) Pub. No.: US 2007/0154408 A1
(43) Pub. Date: Jul. 5, 2007

(54) INHALABLE BIODEGRADABLE MICROPARTICLES FOR TARGET-SPECIFIC DRUG DELIVERY IN TUBERCULOSIS AND A PROCESS THEREOF

(75) Inventors: Himadri Sen, Pune (IN); Suryakumar Jayanthi, Pune (IN); Rakesh Sinha, Pune (IN); Rolee Sharma, Lucknow (IN); Pavan Muttil, Lucknow (IN)

Correspondence Address: SUGHRUE MION, PLLC, 2100 PENNSYLVANIA AVENUE, N.W., SUITE 800, WASHINGTON, DC, 20037, US

(73) Assignee: COUNCIL OF SCIENTIFIC & INDUSTRIAL RESEARCH; LUPIN LIMITED [RESEARCH PARK]

(21) Appl. No.: 11/684,559

(22) Filed: Mar. 9, 2007

Related U.S. Application Data
(62) Division of application No. 10/685,567, filed on Oct. 16, 2003.

(51) Int. Cl. A61K 9/14

(52) U.S. Cl. 424/46

(57) ABSTRACT

The present invention relates to a biodegradable microparticle composition useful for the target specific drug delivery to manage pulmonary tuberculosis, said composition comprising two anti-tuberculosis drugs, and a biodegradable polymer for drug delivery in a ratio of about 1:2 to 2:1, wherein the anti-tubercular drugs are in the ratio of 1:2 to 2:1, also, a process for the preparation of the composition, and lastly, a method of treating pulmonary tuberculosis in a subject, said method comprising administering by inhalation alone or in combination with oral route, pharmaceutically effective amount of the composition to the subject in need thereof, wherein the dosage for inhalation is ranging between 0.5 to 10 mg/kg body weight/day and that for oral route is ranging between 4 to 32 mg/kg body weight/day.

### FIELD OF THE PRESENT INVENTION

The present invention relates to a biodegradable microparticle composition useful for the target specific drug delivery to manage pulmonary tuberculosis, said composition comprising two anti-tuberculosis drugs, and a biodegradable polymer for drug delivery in a ratio of about 1:2 to 2:1, wherein the anti-tubercular drugs are in the ratio of 1:2 to 2:1, also, a process for the preparation of the composition, and lastly, a method of treating pulmonary tuberculosis in a subject, said method comprising administering by inhalation alone or in combination with oral route, pharmaceutically effective amount of the composition to the subject in need thereof, wherein the dosage for inhalation is ranging between 0.5 to 10 mg/kg body weight/day and that for oral route is ranging between 4 to 32 mg/kg body weight/day.

### BACKGROUND AND PRIOR ART REFERENCES OF THE INVENTION

Most infectious diseases are caused by parasitic microorganisms, which reside in specific areas in the human body. The cells present in these areas are infected by these microorganisms thereby resulting in the localization of these disease-causing agents to specific cells.

Macrophages are cells that are part of the human immune system and are present in the liver, lungs, spleen, lymph nodes, thymus, gut, marrow, brain, connective tissue and serous cavities. ^{[1]} Their main functions as a first line defense against infectious, is by phagocystosis of the microorganisms. ^{[2]} But certain facultative or obligate intracellular parasites use macrophages as safe haven. In patients infected by these microorganisms, the macrophages act as reservoirs for them. ^{[3,4]}.

Tuberculosis (TB) is a leading cause of infectious lung disease, and considered the foremost cause of death due to a single microorganism. Tuberculosis has become a significant opportunistic disease among population with a high incidence of acquired immunodeficiency syndrome (AIDS).

The occurrence of TB is most often due to Mycobacterium tuberculosis (MTB) infection, and the lungs are the primary site of infection for the systemic pathogen. Problems created by bacterial infection are linked to their ability to survive and multiply inside the body, especially in the lungs, and to the natural immune response of the infected host.

Tuberculosis, and more importantly pulmonary tuberculosis, is best treated successfully only through a treatment schedule than can achieve sustained drug concentrations for prolonged periods. But sometimes even such methods results in failure. One reason could be that oral administration of drugs may not achieve appreciable concentrations in the cytosol of target cells like macrophages.

Bacteria reaching the deep lung are phagocytized by alveolar macrophages in the first step of pathogenesis. Inside the macrophage the bacteria will either be destroyed, begin replicating, or remain latent indefinitely. If replication is not prevented, the bacilli will multiply and eventually cause the macrophage to rupture.

Current treatments of tuberculosis are limited by their methods of delivery. Persistent, high blood levels of anti tubercular drugs resulting from prolonged oral administration may not be sufficient to kill mycobacteria residing in macrophages.

In order to solve this problem, several investigators have proposed the administration of the drugs in the form of vesicular systems as inhalations ^{[5]} or injectable preparation ^{[6]} as also microparticulate systems for injection. ^{[7,8]}. Recently, O'Hara and Hickey suggested that administration of biodegradable microspheres through the bronchio-pulmonary route for better therapy of tuberculosis. ^{[9 ]}

In order to target the microorganisms present in the macrophages, it is essential to develop a formulation whereby the therapeutic agent can be delivered at high concentration into the macrophage. It is a known fact that even when the drugs are in high concentration in soluble form in the serum, very little of it reach the macrophages. Moreover, macrophages are programmed to phagocytose any foreign particle they encounter thereby making it difficult to build up high intra cellular concentration of the drug in a macrophage. One way to overcome this problem is using carriers for transporting the drugs. Liposomes and microparticles are two types of carriers, which have been widely studied.

Drug doses above those currently administered would present the risk of toxic side effects since the anti-tubercular agents are at their maximum tolerated dose for systemic exposure. Targeting anti-tubercular drug delivery to the lung may increase local therapeutic effect and reduce systemic exposure.

Various researchers have reported the development of dry powder inhalations for pulmonary delivery of drugs ^{[10,11,12]}, Particles delivered to the lungs are rapidly phagocytosed by the alveolar macrophages. ^{[13]}

U.S. Pat. No. 6,264,991 discloses compositions and methods for treating intracellular infections comprising administering a first effective amount of a suitable drug contained in first biocompatible microspheres having a diameter less than 10 microns and a second set of microspheres having a diameter more than 10 microns, to provide continuing systemic release of the drug. The administration of the first microspheres is by intravenous route and the second microspheres by subcutaneous route.

In case of tuberculosis, as the microorganisms reside in the macrophages, the inhaled microparticles have a potential to deliver the drug directly into the macrophages resulting in higher concentrations than when given orally.

Moreover, this may result in the possibility of reduction in dosage amount & frequency or duration of treatment.

Alveolar macrophages having anti-TB drugs in microparticulate system along with mycobacteriurn travel through lymphatic circulation to secondary lymphoid organs. Thus, mycobacteria disseminate not only through the bloodstream, but also to sites where alveolar macrophages traffic. Loading bacteria resident alveolar macrophages with drug containing microparticles leads to transportation of drugs to all the sites where migrating macrophages go thereby mimicking the course of spread of bacteria. As a result sufficient drug concentration may also be achieved in the various tissues where bacteria tend to migrate. Due to the presence of a biodegradable polymer, controlled release of drugs may be obtained, thereby prolonging the duration of action.

Following are the Advantages of Noninvasive (Inhalable) Biodegradable Microparticles:

a) Targeting the primary site of tuberculosis infection i.e. lungs.
b) Targeting mycobacterium infected macrophages.
c) Rate controlled release of drugs using selective polymer, their ratios and microparticulate size.
d) Substantial increase of drug concentrations within macrophages, thereby decreasing systemic exposure of drugs resulting in reduced potential side effects.

Tuberculosis treatment being a long-term schedule, these advantages can result in greater patient compliance along with better bioavailability.

Pharmaceutical Research Vol-18 No. 10, October 2001 discloses Rifampicin and Isoniazid combinations which is known to form adduct which might result in reduced bio-availability and efficacy of these drugs. Further high concentration of polymers as much as 3 times w/w of drugs have been used. The instant invention relates to biodegradable, inhalable microparticles containing compatible anti-tubercular drug combination. As the present invention uses lesser quantities of the polymer, the cost of the therapy will also be cheaper.

The U.S. Pat. No. 6,264,991 refers to the treatment of tuberculosis using one or two drugs. However, the dosage and ratios of the constituents are totally distinct. These change in the dosage, size of the particles, and route of administration are critical for the desired results. Further, the published article of the inventors in Pharmaceutical Research Vol 18, No 10 October 2001 refers to the management of tuberculosis. However, the drugs used are distinct from that of the instant Application. The ratio of the polymer and the drug is critica l for the functioning of the composition. Also, ratio of drugs is critical for the desired results. Most importantly, there is no adduct formation in the case of instant Application.

Object of the Present Invention:

The main object of the present invention is to develop an inhalable, biodegradable microparticle composition comprising one or more anti-tubercular drugs for the target specific drug delivery to manage pulmonary tuberculosis.

Another objective of the present invention is to provide a method of enhancing the efficacy of anti-tubercular drugs by administering them through inhalation route. Another main invention of the present invention is to develop a method of treating pulmonary tuberculosis in a subject, said method comprising administering by inhalation alone or in combination with oral route, pharmaceutically effective amount of composition comprising biodegradable inhalable microparticles useful for the target specific drug delivery to manage Pulmonary tuberculosis containing two anti-tubercular drugs and biodegradable polymer in the drug delivery in the ratio of 1:2 to 2:1 to the subject in need thereof, wherein the anti-tubercular drugs are in the ratio of 1:2 to 2:1.

Still another object of the present invention is to develop a method to target alveolar macrophages.

Still another object of the present invention is to develop a method of treating tuberculosis, which helps the drug reach the site in high amount consistently.

Still another object of the present invention is to develop a method to help reduce the amount of drug required for the treatment of tuberculosis.

Still another object of the present invention is to develop a process for the preparation of a biodegradable microparticle composition comprising one or more anti-tubercular drugs, and a biodegradable polymer for drug delivery.

### SUMMMARY OF THE PRESENT INVENTION

The present invention relates to a biodegradable microparticle composition useful for the target specific drug delivery to manage pulmonary tuberculosis, said composition comprising two anti-tuberculosis drugs, and a biodegradable polymer for drug delivery in a ratio of about 1:2 to 2:1, wherein the anti-tubercular drugs are in the ratio of 1:2 to 2:1, also, a process for the preparation of the composition, and lastly, a method of treating pulmonary tuberculosis in a subject, said method comprising administering by inhalation alone or in combination with oral route, pharmaceutically effective amount of the composition to the subject in need thereof, wherein the dosage for inhalation is ranging between 0.5 to 10 mg/kg body weight/day and that for oral route is ranging between 4 to 32 mg/kg body weight/day.

### DETAILED DESCRIPTION OF THE PRESENT INVENTION

Accordingly, the present invention relates to a biodegradable microparticle composition useful for the target specific drug delivery to manage pulmonary tuberculosis, said composition comprising one or more anti-tuberculosis drugs, and a biodegradable polymer for drug delivery in a ratio of about 1:2 to 2:1, wherein the anti-tubercular drugs are in the ratio of 1:2 to 2:1. Also, the invention relates to a process for the preparation of the composition, and lastly, a method of treating pulmonary tuberculosis in a subject, said method comprising administering by inhalation alone or il combination with oral route, pharmaceutically effective amount of the composition to the subject in need thereof wherein the dosage for inhalation is ranging between 0.5 to 10 mg/kg body weight/day and that for oral route is ranging between 4 to 32 mg/kg body weight/day.

In still another embodiment of the present invention, wherein a biodegradable microparticle composition useful for the target specific drug delivery to manage pulmonary tuberculosis, said composition comprising one or more anti-tuberculosis drugs, and a biodegradable polymer for drug delivery.

In still another embodiment of the present invention, wherein the drugs are in the ratio of about 1:1.

In still another embodiment of the present invention, wherein the ratio of drug to polymer is about 1:1.

In still another embodiment of the present invention, wherein the drugs are selected from a group comprising Rifabutin, Rifapentine, Rifampicin, Isoniazid, pyrazinamide and ethambutol.

In still another embodiment of the present invention, wherein the drugs are in preferred combination of Rifabutin and Isoniazid.

In still another embodiment of the present invention, wherein the polymer is selected from a group comprising polyglycolic acid, polylactic acid, poly(lactic acid-co-glycolic acid), polysebacic anhydride, and polycaprolactone and mixtures thereof.

In still another embodiment of the present invention, wherein the preferred polymer is polylactic acid.

In still another embodiment of the present invention, wherein the size of the biodegradable microparticles is ranging between 1-15 micron.

In still another embodiment of the present invention, wherein at least 90% of the microparticles are of size less than 10 microns.

In still another embodiment of the present invention, wherein a method of treating pulmonary tuberculosis in a subject, said method comprising administering by inhalation alone or in combination with oral route, pharmaceutically effective amount of composition comprising of bio-degradable inhalable microparticles useful for the target specific drug delivery to manage Pulmonary tuberculosis containing one or more anti -tubercular drugs and bio-degradable polymer in the drug delivery in the ratio of 1:2 to 2:1 to the subject in need thereof, wherein the anti-tubercular drugs are in the ratio of 1:2 to 2:1.

In still another embodiment of the present invention, wherein the said method targets alveolar macrophages.

In still another embodiment of the present invention, wherein said method shows no deleterious effect on the subject.

In still another embodiment of the present invention, wherein said subject is an animal including humans.

In still another embodiment of the present invention, wherein the dosage for inhalation is ranging between 0.5 to 10 mg/kg body weight/day.

In still another embodiment of the present invention, wherein the dosage for oral administration is ranging between 4 to 32 mg/kg body weight/day.

In still another embodiment of the present invention, wherein said method helps drug reach the site in high amount consistently.

In still another embodiment of the present invention, wherein said method helps reduces the amount of drug required for the treatment of tuberculosis.

In still another embodiment of the present invention, wherein a process for the preparation of a microparticle composition comprising one or more anti-tuberculosis drugs, and a biodegradable polymer for drug delivery, said process comprising steps of;
dissolving the drug (s) in an aqueous solvent preferably, alcohol or any other suitable solvent to obtain a solution;
dissolving the polymer in dichloromethane or any other suitable solvent to obtain another solution,
mixing the aforementioned solutions to obtain the final solution, and
spray-drying the final solution to obtain the microparticles.

In still another embodiment of the present invention, wherein the microparticles are of the size ranging between 1-15 micron.

In still another embodiment of the present invention, wherein at least 90% of the microparticles are of size less than 10 microns.

In still another embodiment of the present invention, wherein the drugs are selected from a group comprising Rifabutin, Rifapentine, Rifampicin, Isoniazid, pyrazinamide and ethambutol.

In still another embodiment of the present invention, wherein the polymer is selected from a group comprising polyglycolic acid, polylactic acid, poly(lactic acid-co-glycolic acid), polysebacic anhydride, polycaprolactone or mixtures thereof.

### BRIEF DESCRIPTION OF THE PRESENT INVENTION

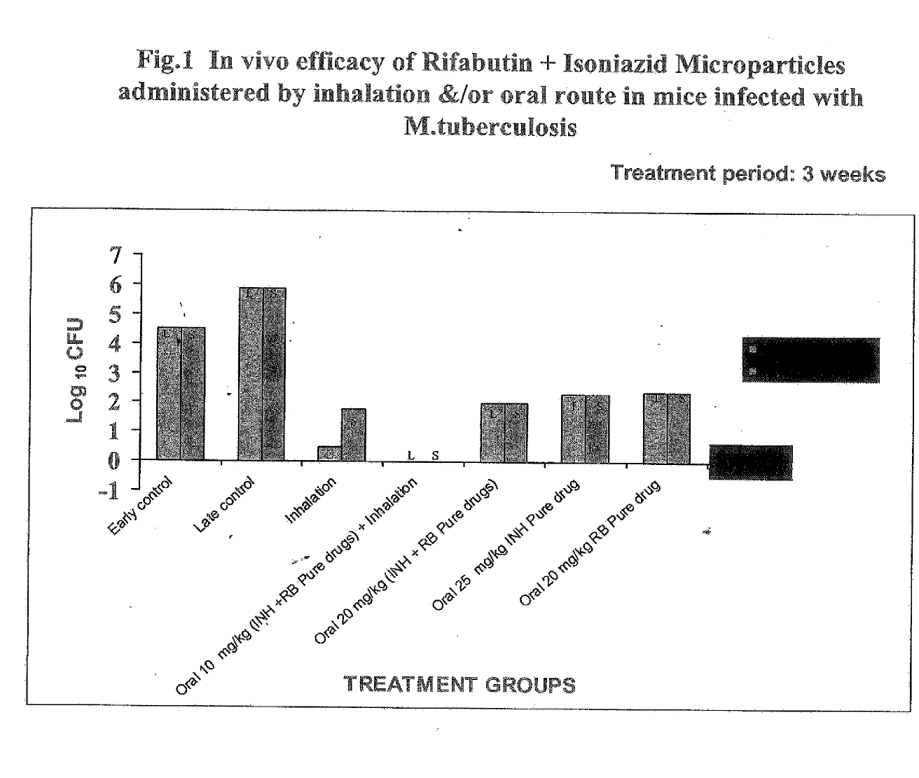

FIG. 1 shows the results after treatment of tuberculosis with the composition of the instant Application for 3 weeks.

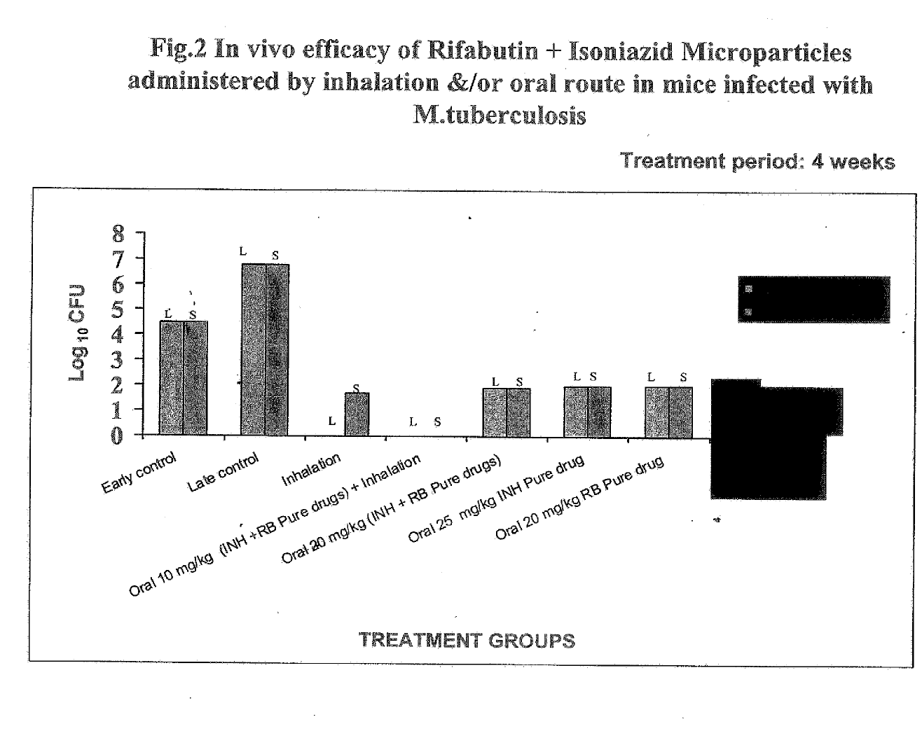

FIG. 2 shows the results after treatment of tuberculosis with the composition of the instant Application for 4 weeks.

Typically anti-tubercular drugs such as Rifampicin, Isoniazid, Ethambutol and Pyrazinamide are given orally in the range between 4-32 mg/kg body weight/day.

TABLE 1
Lists the dosage ranges of the anti-TB drugs per day (Lower
dosage is for a subject of body weight less than 50 kgs)

| ANTI-TB DRUGS ORAL DOSE | |
|---|---|
| (WHO APPROVED) | PER KG BODY WT. |
| 1. RIFAMPICIN - 450 mg to 600 mg/day | Range for all drugs |
| 2. ISONIAZID - 225 mg to 300 mg/day | is between 4 to 32 |
| 3. ETHAMBUTOL - 825 mg to 1100 mg/day | mg/kg body weight/day |
| 4. PYRA ZINAMIDE - 1200 mg to 1600 | |
| mg/day | |

TABLE 2
Lists the dosage ranges of the anti-TB drugs
on the basis of per kg body weight
ANTI-TB DRUGS ON THE BASIS OF PER KG BODY WT.

1. RIFAMPICIN - 9 to 12 mg/kg body weight
2. ISONIAZID - 4.5 to 6 mg/kg body weight
3. ETHANBUTOL - 16.5 to 22 mg/kg body weight
4. PYRAZINANIDE - 24 to 32 mg/kg body
5. RIFABUTIN - 300 mg/day is equivalent to 6 mg/kg body weight

One aspect of the invention provides a method for the preparation of microparticles of one or more therapeutic agents along with a biodegradable polymer.

In a preferred embodiment the microparticles contain one or more anti-tubercular drugs. In a preferred embodiment, the anti-tubercular drugs are selected from the group consisting of Rifampicin, Rifabutin, Rifapentine, Isoniazid, pyrazinamide and ethambutol.

In another preferred embodiment the ratio of Rifabutin to Isoniazid is 1:2 to 2:1 and the drugs to polymer ratio is also 1:2 to 2:1.

In a preferred embodiment, the biodegradable polymer is selected from the group comprising polyglycolic acid, polylactic acid, poly(lactic acid-co-glycolic acid) polysebacic anhydride and polycaprolactone or mixtures thereof . In another preferred embodiment, the biodegradable polymer is polylactic acid.

The composition of the invention may be formulated using techniques generally known in the art of microparticle synthesis. In a preferred embodiment, the microparticles are made by the spray—drying technique. In one particular embodiment, the microparticles have a size ranging from 1 to 15 microns with at least 90% of the microparticles having a size below 10 microns.

To accomplish the objects of the present invention, the microparticles can be administered in inhalable form for pulmonary delivery. The methods and composition of this invention can be used to treat pulmonary tuberculosis in any animal.

Moreover, the invention also describes methods of combining two alternate routes of drug administration to achieve significant reduction in bacterial levels as compared to conventional therapy.

Methods for making specific and preferred compositions of the present invention are described in the example below. However, these examples should not be construed to limit the scope of the invention.

Preparation of Rifabutin+Isoniazid Microparticles

Isoniazid (750 mg) was dissolved in methanol (8 mL) by warming lightly. Poly-lactic acid (1.5 g) and Rifabutin (750 mg) was dissolved in dichloromethane (90 ml). Both solutions were mixed using 2 mL portions of methanol to wash the Isoniazid solution. The solution was spray dried using Buchi Spray Dryer. The solution feed rate was adjusted to 5mL/minute at a setting of 13% with the inlet temperature of 52° C. and aspirator at 70%. The atomizing air was supplied at 1.6 kg/cm². The microparticles were analysed for the drug(s) content and the particle size was determiped using Malvern Particle size Analyzer Model Mastersizer 2000.

### EXAMPLE NO. 1

In Vivo Studies on Mice

Preparation of Microparticles/Drug Solutions

a. Rifabutin+Isoniazid Microparticles

The above microparticles were prepared as described earlier. Particle size analysis of the microparticles showed 90% below 10 microns.

b. Rifabutin solution

Rifabutin solution was prepared for oral administration using 10% DMSO in water.

c. Isoniazid solution

Isoniazid solution was prepared for oral administration using water.

Infection & Treatment

Swiss Albino Mice 4-6 weeks old weighing 18-22 gms were used for the study. The mice were infected with 10⁷ CFUs/0.2 mL of Mycobacterium tuberculosis H₃₇Rᵥ strain by intravenous route. All infected animals were distributed in 8 groups of six mice each. Infected mice were treated with the drug fonnulations 24 hours post infection as below:
Group 1—Rifabutin+Isoniazid microparticle by inhalation (15-20 mg/30 sec /mouse)
Group 2—Rifabutin+Isoniazid microparticle by inhalation (15-20 mg/30 sec /mouse) along with oral administration of free Rifabutin (5 mg/kg) and free Isoniazid (5 mg/kg)
Group 3—Oral administration of free Rifabutin (10 mg/kg) and free Isoniazid (10 mg/kg)
Group 4—Oral administration of free Isoniazid (25 mg/kg)
Group 5—Oral administration of free Rifabutin (20 mg/kg)
Group 6—Early infection control
Group 7—Late infection control
Group 8—Negative (Vehicle control)
   Treatment Schedule

All animals were treated once a day, 5 days per week for 3 weeks/4 weeks.

In groups treated by inhalation, the microparticles were delivered by generating aerosols in a tube with a rubber teat.

Early controls were sacrificed 24-hours post infection i.e. at the start of the treatment.

All animals treated as well as untreated late controls were sacrificed 3 days post treatment to enumerate the mycobacterial load in the organs. Viable tubercle bacilli from the target organs i.e, lung and spleen were enumerated by plating out the different dilution on individual organ homogenates on the Middle brook 7H 10 media plates. The plates were incubated at 30° C. for 3-4 weeks. Mycobacterial load in each organ was determined by counting the colonies on the plates.

Observation:

After Treatment for 3 Weeks

In the present study no growth was seen in lungs of 4 out of 6 animals when the formulation was administered by inhalation route alone (Group 1). The spleens of the animals however showed the presence of low levels of viable bacilli.

Lungs and spleen were found to be free of infection in all the mice treated by inhalation in combination with half the oral dose of pure drugs (Group 2). Significant reduction (2 log) in the bacterial load was observed in both the organs (lung, spleen) in the animals treated by oral route with either combination of pure drug or alone.

After Treatment for 4 Weeks

Lungs in all the mice were found to be free of infection when treated by inhalation route alone (Group 1). The spleens of the animals however showed the presence of low levels of viable bacilli.

Lungs and spleen were found to be free of infection in all the mice treated by inhalation in combination with half the oral dose of pure drugs (Group 2). Significant reduction (2 log) in the bacterial load was observed in both the organs (lung, spleen) in the animals treated by oral route with either combination of pure drug or alone.

The results indicate that treatment of M.tuberculosis infected animals by inhalation route using microparticles in combination with oral route (half the dose) results in faster clearance of tubercle bacilli from the organs.

FIGS. 1 & 2 show the results after treatment for 3 and 4 weeks respectively.

Normally all the 4 Anti-Tubercular drugs (Rifampicin, Isoniazid, Ethambutol and Pyrazinamide) are administered to tuberculosis patients for a period of at least 2 months, before switching over to Rifampicin and Isoniazid for at least 4 months. In the present invention, lungs and spleen were found to be free of infection in all the mice by using 2 drugs only (Rifabutin and Isoniazid).

Further, the lesser is the number of drugs in the composition, better is the performance.

### BIBLIOGRAPHY

1. B. Vemon-Robert, The macrophage, in: R. J. Harrison, R. M. H. Mcminn (Eds.), Biological structure and function, Vol 2, Cambridge University Press, Condon, 1972.
2. D. P. Speert, in: C. E. Lewis, J. O'D. McGee (Eds.)., The Natural Immune System: The Macrophage, Oxford University Press, New York, 1992, PP. 215-263.
3. J. Stewart, D. M. Weir, Immunity in Bacterial infections, in: D. Greenwood, R. Slack, J. Pentherer (Eds.), 14^{th} Edition, Medical Microbiology, Churchill Livingstrong, Edinburgh, 1942, PP 195-200.
4. G. R. Donowitz. Tissue-directed antibiotics and intracellular parasites: Complex interaction of phagocyte, pathogens and drugs, Clin. Infect. Dis. (1994) 926-930.
5. Y. N. Kurunov, P. A. Filimonov, A. V. Svistelnik et al. Efficacy of liposomized antibacterial drugs in inhalation therapy of experimental tuberculosis. Probl. Tuberk 1:38-40 (1995).
6. P. Deol, G. K. Khuller, and K. Joshi. Therapeutic efficacies of isonazid and rifampin encapsulated in lung-specific stealth liposomes against mycobacterium tuberculosis infection induced in mice. Antimicrobial agents chemother 41:1211-1214 (1997)
7. D. C. Quenelle, J. K St as, G. A. Winchester et. al. Efficacy of microencapsulated rifampin in mycobacterium tuberculosis—infected mice. Antimicrobial Agents Chemother. 43:1144-1151 (1999)
8. E. L. Barrow, G. A. Winchester, J. K. Staas et.al. Use of microsphere technology for targeted delivery of rifampin to mycobacterium tuberculosis—infected macrophages. Antimicrobial Agents Chemother. 42:2682-2689.
9. P. O'Hara and A. J. Hickey. Respirable PLGA microspheres containing rifampicin for the treatment of tuberculosis: manufacture and characterization. Pharm. Res. 17:955-961 (2000)
10. Y. Kawashima, T. Serigano, T. Hino et.al. surface-modified antiasthmatic drug powder aerosols inhaled intratracheally reduce the pharmacologically effective dose. Pharm. Res. 15:1753-1759 (1998).
11. J. S. Patton. Deep-lung delivery of proteins. Modern Drug Discover 2:19-28 (1999)
12. R. J. Malcolmson and J. K. Embleton. Dry powder formulations for pulmanory delivery. Pharma. Sci.Tech.index 1:394-398 (1998).
13. C. Evora, I. Soriano, R. A. Rogers et. al. Relating the phagocytosis of microparticles by alveolar macrophages to surface chemistry: The effect of 1,2-dipalmitoyl phosphatidylcholine. J. Control. Release 51:43-152 (1998).

1-8. (canceled)

9. A method of treating pulmonary tuberculosis in a subject, said method comprising administering by inhalation alone or in combination with oral route, pharmaceutically effective amount of composition of a biodegradable inhalable microparticle composition to the subject in need thereof.

10. The method as claimed in claim 9, wherein the said method targets alveolar macrophages.

11. The methods claimed in claim 9, wherein said subject is an animal including humans.

12. The method as claimed in claim 9, wherein the dosage for inhalation is ranging between 0.5 to 10 mg/kg body weight/day.

13. The method as claimed in claim 9, wherein the dosage for oral administration is ranging between 4 to 32 mg/kg body weight/day.

14. The method as claimed in claim 9, wherein said method helps drug reach the site in high amount consistently.

15. The method as claimed in claim 9, wherein said method helps reduces the amount of drug required for the treatment of tuberculosis.

16. The method as claimed in claim 9, wherein said method shows 2 log reduction in the bacterial load.
